(19) United States
(12) Patent Application Publication
(10) Pub. No.: US 2015/0264884 A1
(43) Pub. Date: Sep. 24, 2015

(54) GENETICALLY MODIFIED SALT WATER TOLERANT PLANT

(71) Applicant: John Grossheim, Cincinnati, OH (US)

(72) Inventor: John Grossheim, Cincinnati, OH (US)

(21) Appl. No.: 14/664,753

(22) Filed: Mar. 20, 2015

Related U.S. Application Data
(60) Provisional application No. 61/955,851, filed on Mar. 20, 2014.

(51) Int. Cl. A01H 5/10; C12N 15/82

(52) U.S. Cl. CPC A01H 5/10; C12N 15/8273

(57) ABSTRACT

Genetically modified plants are described which stably express the Salt Tolerance Protein that improves the salt tolerance of the plant when compared with to a corresponding genetically unmodified wild-type plant cells or wild-type plants are described. Also described are methods of increasing the salt tolerance of a plant by stably introducing into the plant a nucleic acid encoding the Salt Tolerance Protein.

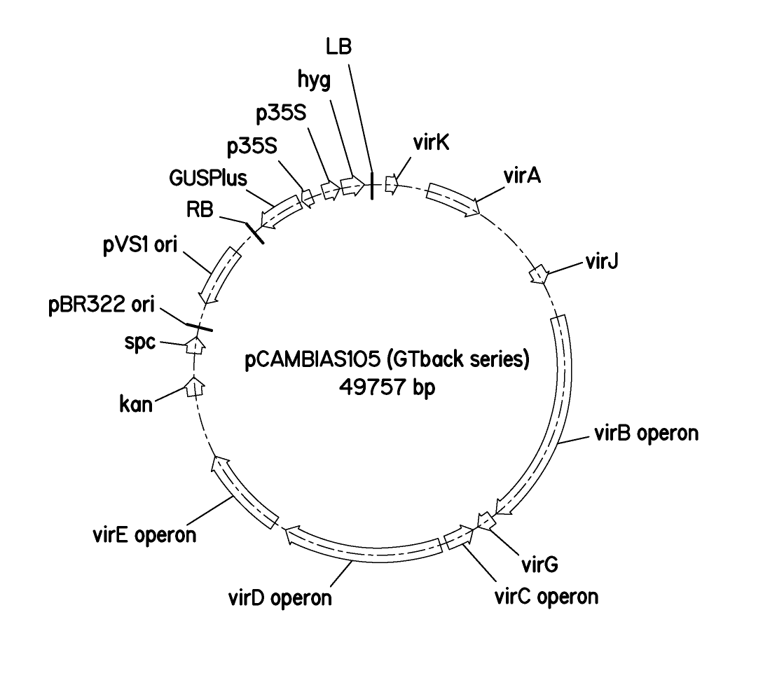

### RELATED APPLICATION

The present application claims the benefit of U.S. Ser. No. 61/955,851 filed Mar. 20, 2014, the disclosure of which is hereby incorporated herein by reference in its entirety.

### FIELD

The present invention relates to a genetically modified salt water tolerant plant, and more particularly to a genetically modified salt water tolerant rice plant.

### BACKGROUND

Many regions of the world do not have access to sufficient quantities fresh water for cultivating crops. Climate change is exacerbating this problem by reducing the availability of fresh water. Water high in salinity, such as sea water, is plentiful. However, most crops that are desired for cultivation cannot grow in high salinity conditions, such as when irrigated with sea water. Crops that are tolerant to high salinity conditions are needed.

Mangrove plants thrive in the relatively high saline environment of ocean water. Sesuvium Portulacastrum, commonly referred to as shoreline sea purslane, is one such mangrove plant. A group of researchers in Malaysia has recently isolated gene SRTG152 from S. portulacastrum. The protein product from the SRTG152 gene contributes to the salt water tolerance to S. portulacastrum.

### SUMMARY

Aspects of the invention are directed to genetically modifying plants to improve the salt tolerance of the plant. The resulting genetically modified plants are capable of being cultivated in soil having high salinity or when irrigated with high salinity water, such as sea water. An aspect of the invention is directed to a plant that has been genetically modified to have improved tolerance to salt. In particular, the plant has been genetically modified to express an exogenous nucleic acid encoding the SRTG152 gene having SEQ ID NO: 1. The nucleic acid for the SRTG152 gene encodes the Salt Tolerance Protein that improves the salt tolerance of the plant. The Salt Tolerance Protein has SEQ ID NO: 2.

Another aspect of the invention is directed to methods of improving the salt tolerance of a plant by the stable introduction of a nucleic acid directed to the SRTG152 gene to result in the expression of the Salt Tolerance Protein and an increase in the ability of the plant to tolerate elevated salt concentrations.

Various additional objectives, advantages, and features of the invention will be appreciated from a review of the following detailed description of the illustrative embodiments taken in conjunction with the accompanying drawings.

### BRIEF DESCRIPTION OF THE DRAWINGS

The accompanying drawing, which is incorporated in and constitute a part of this specification, illustrates an embodiment of the invention and, together with a general description of the invention given above, and the detailed description given below serve to explain the invention.

The FIGURE shows a map of the pCAMBIA5105 Ti-Plasmid.

### DETAILED DESCRIPTION

A first aspect of the present invention relates to a plant cell or a plant which is genetically modified, the genetic modification leading to the stable expression of Salt Tolerance Protein that improves the salt tolerance of the plant when compared with to a corresponding genetically unmodified wild-type plant cells or wild-type plants.

The genetic modification can here be any genetic modification which leads to the stable expression of Salt Tolerance Protein that improves the salt tolerance of the plant when compared with to a corresponding genetically unmodified wild-type plant cells or wild-type plants.

The term “wild-type plant cell” means, in connection with the present invention, that these are plant cells which served as a starting material for the production of the plant cells according to the invention, i.e. their genetic information, apart from the genetic modification introduced, corresponds to that of a plant cell according to the invention.

In connection with the present invention, the term “wild-type plant” means that these are plants which served as a starting material for the production of the plants according to the invention, i.e. their genetic information, apart from the genetic modification introduced, corresponds to that of a plant according to the invention.

The term “corresponding” means, in connection with the present invention, that on comparison of a number of articles, the articles in question which are being compared with one another were kept under identical conditions. In connection with the present invention, the term “corresponding” in connection with a wild-type plant cell or wild-type plant means that the plant cells or plants which are being compared with one another were grown under identical culture conditions and that they have an identical (cultivation) age.

The improvement of salt tolerance of the genetically modified plant can be determined by, for example, comparing plant cells or plants expressing the Salt Tolerance Protein with plant cells or plants not expressing the Salt Tolerance Protein when cultivated under identical conditions in the presence of elevated levels of salt. The comparison may include, for example, plant or cell survival, growth rate, seed or fruit production, general health, and combinations thereof. Elevated levels of salt include for water include water having salts at a concentration of about 0.1% by weight or more, or from about 0.1% by weight to about 4% by weight, or from about 0.3% by weight to about 3.8% by weight, or from about 1% to about 3.8%, or about 3% to about 3.8%. or about 3.2% to about 3.7%.

The terms “stably express” and “stable expression” of Salt Tolerant Protein means expression of the protein to a level sufficient to improve the salt tolerance of the genetically modified plant or cell over the course of at least a portion of the life cycle of the plant or cell.

In view of the aspects above, an embodiment of the invention is directed to a plant or plant cell that has been genetically modified to increase the plant's or plant cell's tolerance to salt. In particular, cells of the plant are genetically modified with the stable introduction of a nucleic acid directed to the SRTG152 gene. The SRTG152 gene, shown in the Sequence Listing below as SEQ ID NO: 1, encodes the Salt Tolerance Protein that improves the salt tolerance of the plant. The Salt Tolerance Protein has the amino acid sequence set forth in the Sequence Listing below as SEQ ID NO: 2.

In embodiments of the invention, the plant is a food plant. For example, rice is a widely grown highly nutritious food plant that is grown around the world. As such, in an embodiment, the food plant is a rice plant, such as Oryza sativa. In some embodiment other types of plants will also benefit from improved salt tolerance. For example, other food plants such as grains, leafy plants, vegetables, fruits, tubers, legumes, may be genetically modified to improve their salt tolerance as described herein. Additionally, non-food plants, such as grasses, flowers, bushes, etc. may also be genetically modified to improve their salt tolerance as described herein that can be used in landscaping, with golf courses and the like.

Another embodiment of the invention is directed to methods of improving the salt tolerance of a plant or plant cell by the stable introduction of a nucleic acid directed to the SRTG152 gene to result in the expression of the Salt Tolerance Protein.

The SRTG125 gene may be obtained from the common mangrove plant Sesuvium Portulacastrum, commonly referred to as shoreline sea purslane. For example, mRNA encoding the SRTG152 gene may be obtained from samples of S. portulacastrum such as with the TRIReagent kit per the instructions included with the kit. The extracted RNA may be amplified with the polymerase chain reaction (PCR) method utilizing primers for the SRTG152 gene. The SRTG152 may be spliced out of the amplified RNA with restriction enzymes. The presence of the SRTG152 gene in the PCR product may be confirmed with standard techniques such as Northern blotting. The PCR product is then converted to cDNA having a promoter that will allow insertion into the vector and allows for expression in both the cloning bacteria (such as E. coli) and the inoculating agrobacteria (such as A. tumefaciens).

In embodiments of the method, the SRTG152 gene is introduced into a Ti plasmid that includes the S35 promoter region, such as pCAMBIA5105 plasmid. First, the mRNA for the SRTG152 gene is converted into cDNA via rtPRC with primers that insert the S35 promoter region adjacent the gene. In the exemplary embodiment, the forward primer for the rtPCR has SEQ ID NO: 3 and corresponds with the nucleic acid sequence at the 3′ end of the SRTG152 gene. And, the reverse primer has SEQ ID NO: 4 and is complementary to the last three nucleic acids at the 5′ end of the SRTG152 gene. The reverse primer also includes additional nucleic acids that encode the S35 promoter region. The cDNA is introduced into the plasmid at the S35 promoter regions of the plasmid.

The plasmid is used to transform E coli utilizing routine transformation techniques where the gene is amplified in a gene cloning step. The amplified gene product is collected and the SRTG152 gene with adjacent promoter region and spliced out of the gene with restriction enzymes. The spliced out amplified gene is then inserted into a plasmid having a suitable promoter region (e.g., S35 promoter region) and that is suitable for use with an agrobacterium capable of inoculating the plant of interest, such as Agrobacterium tumefaciens. An exemplary plasmid for use with agrobacterium is the pCambia1300 plasmid. The transformed agrobacterium is used to inoculate the plant of interest. For example, the plant may be inoculated with the transformed agrobacterium by injection, by disrupting the surface of the plant and applying the agrobacterium, or by other methods known in the art. The transformed agrobacterium will form a crown gall at the inoculation sight. Nucleic acids will be collected from the crown gall to confirm the expression of the SRTG152 gene. The tissue from the crown gall of plants expressing the SRTG152 gene is then used in routine methods form plants expressing the SRTG152 gene and correspondingly, the Salt Tolerance Protein. The expressed Salt Tolerance protein increases the tolerance of the plant to elevated levels of salt in soil and water.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method and illustrative examples shown and described. Accordingly, departures may be from such details without departing from the scope or spirit of the general inventive concept.

What is claimed is:

1. A genetically modified plant exhibiting tolerance to salt comprising an exogenous nucleic acid expressing a protein encoded by the SRTG152 gene.

2. The genetically modified plant of claim 1 wherein SRTG152 gene is encoded by SEQ ID NO: 1.

3. The genetically modified plant of claim 1 wherein the protein is the Salt Tolerance Protein having SEQ ID NO: 2.

4. The genetically modified plant of claim 1 wherein the plant is a food plant.

5. The genetically modified plant of claim 4 wherein the food plant is a rice plant.

6. The genetically modified plant of claim 5 wherein the rice is Oryza sativa.

7. The genetically modified plant of claim 1 wherein the plant is capable of growing in salt water having a salinity of at least 0.1% by weight.

8. The genetically modified plant of claim 1 where in the plant is capable of growing in salt water having a salinity of ranging from about 0.1% by weight to about 4% by weight.

9. A method of increasing the tolerance of a plant to salt comprising stably introducing an exogenous nucleic acid encoding the SRTG152 gene into a cell of the plant such that the plant expresses the Salt Tolerance Protein.

10. The method of claim 9 wherein the nucleic acid encoding the SRTG152 gene has SEQ ID NO: 1.

11. The method of claim 9 wherein said nucleic acid further includes a S35 promoter region adjacent an end of the SRTG152 gene.

12. The method of claim 9 wherein the Salt Tolerance Protein has SEQ ID NO: 2.

13. The method of claim 9 wherein said plant is a food plant.

14. The method of claim 13 wherein said food plant is rice.

15. The method of claim 14 wherein said food plant is Oryza sativa.

16. The method of claim 9 wherein said nucleic acid further includes a S35 promoter adjacent an end of the SRTG152 gene.

17. The method of claim 9 wherein said nucleic acid is included in a pCAMBIA5105 vector.

18. A plant produced by the method of claim 9.


SEQUENCE LISTING (4 sequences)

SEQ ID NO: 1 - DNA, 1291 bp, Sesuvium portulacastrum
atgcgggaaggttggagaaaatataagctgctatccagtctaagctcttctcctcaacttcattttcttacaagggccatggcatcagatgataaaccagaggtagctgaaagggttaccagagggaaagatcacgaggaggaaaaagaggaagataaaggtggatttatagacaaggtgaaggattttattcaggatattggtgagaagatagagggagcaattggatttggtaagcctactgcagatgtttccggggttcattttcctcatattgacctccataaggcagaggttattgttgatgttcttgtgaaaaaccccaatccagtactaattcctcttatcgacataaactatttgattgaaagtgatggaaggaagctggtttctggattgattcgtgatgctggaacaatccgtgctcatggctccgagactgtcaagatacctgtcaatgtgatttatgattacattaagagcacttatgaggatatcaagcctggaagcattattccttataatgttaaggttgatctgattatagatgttccagttattggaaggatcaccataccccttcagaaaaccggtgaaatccctgtgccatacaaacccgacatagatgtcgagaaaatacactttgagaggttctcttttgaggagactattgcaacactcaagctcaaactagagaacaagaatgactttgacttgggtcttaatgctttggactatgaggtttggctaggtgacgagaacattggaggtgctgagctccagaaatctgctaaaattgagaaaaatggtatcacccatatggaccttccaatctcatttaggcccaaggattttgggtcggcactctgggatatgattaggggaagtggcactggttacaccatgaagggaaatattgacgtggatacaccctttggagtgatgaagcttccaatcagcaaagagggtggaacaacccgccttaagaaaaagaaggatgatgggagctatgacgatgatgatgacgaggattagatcaaatcatctggagatgccatcttgtactcaatacagctagtcaatttgtatgagactttacaaatagttgcacaatgaagcgtccttttagtttctatggcgcttacaaagccatcgggtataataagcatcattgctgctgttagattgtttgtattattgcggatcacaactacagcaatcgtttgtatcgattgagctataacttactactatgatgattttagacataacttatgatttggttttcgtg

SEQ ID NO: 2 - protein, 344 aa, Sesuvium portulacastrum
MREGWRKYKLLSSLSSSPQLHFLTRAMASDDKPEVAERVTRGKDHEEEKEEDKGGFIDKVKDFIQDIGEKIEGAIGFGKPTADVSGVHFPHIDLHKAEVIVDVLVKNPNPVLIPLIDINYLIESDGRKLVSGLIRDAGTIRAHGSETVKIPVNVIYDYIKSTYEDIKPGSIIPYNVKVDLIIDVPVIGRITIPLQKTGEIPVPYKPDIDVEKIHFERFSFEETIATLKLKLENKNDFDLGLNALDYEVWLGDENIGGAELQKSAKIEKNGITHMDLPISFRPKDFGSALWDMIRGSGTGYTMKGNIDVDTPFGVMKLPISKEGGTTRLKKKKDDGSYDDDDDED

SEQ ID NO: 3 - DNA, 17 bp, Artificial Sequence
  Forward Primer for PCR.
tacgcccttccaacctc

SEQ ID NO: 4 - DNA, 27 bp, Artificial Sequence
  Reverse Primer for PCR.
cacgaaaaccaaatcataagttatgtc